(19) United States
(12) Patent Application Publication
(10) Pub. No.: US 2023/0338593 A1
(43) Pub. Date: Oct. 26, 2023

(54) ULTRA-VIOLET STERILIZATION SYSTEM FOR A DEVICE SURFACE

(71) Applicant: Martin Kuster, Walchwil (CH)

(72) Inventor: Martin Kuster, Walchwil (CH)

(21) Appl. No.: 17/762,074

(22) PCT Filed: Sep. 16, 2020
(86) PCT No.: PCT/IB2020/058585

§ 371 Date: Mar. 20, 2022

Related U.S. Application Data
(60) Provisional application No. 62/903,716, filed on Sep. 20, 2019.

(51) Int. Cl. A61L 2/10; A61L 2/26

(52) U.S. Cl. CPC A61L 2/10; A61L 2/26; A61L 2202/11; A61L 2202/16

(57) ABSTRACT

A device includes a display and a sterilization system. The sterilization system includes an ultra-violet (UV) light source that is configured to emit UV light. The sterilization system is configured to selectively direct UV light from the UV light source into the display. In some aspects, the UV light source includes at least one UV light emitting diode. In various aspects, the device is a mobile phone.

### REFERENCE TO RELATED APPLICATION

This application claims the benefit of U.S. Provisional Patent Application No. 62/903,716, filed on Sep. 20, 2019 and entitled ULTRA-VIOLET STERILIZATION SYSTEM FOR A DEVICE SURFACE, the content of which is hereby incorporated by reference in its entirety.

### FIELD OF THE INVENTION

Embodiments of the present invention relate to sterilization of device surfaces, and, more particularly, to sterilization systems for devices.

### BACKGROUND

Many devices, particularly devices with screens, glass surfaces, or other similar surfaces, have surfaces that are commonly are exposed to and have bacteria, germs, and other contaminants on surfaces of the device. Such devices may be portable devices (such as mobile phones) or devices integrated into a larger component (such as navigation touchscreens). Such devices may be used continuously through a day in a variety of environments for both personal and work purposes.

The most common way to control the bacteria and other contaminants on the surfaces of such devices is to use antibacterial wipes or other similar cleaning surfaces. However, such wipes may leave streaking on the surfaces of the device and rely on chemical solutions to perform the cleaning. Moreover, if a person does not happen to have such wipes readily available, they are unable to clean the device. Another way to clean a device is by using a dry towel. However, a dry towel does not eliminate bacteria and viruses, but rather just removes visible dirt. Cleaning with a wet wipe may kill bacteria and viruses if an appropriate fluid is used, but a risk of using fluid is that the fluid may get into the device. Another downside of relying on a fluid to clean the device is that the user mush have the appropriate chemical with them, and some people do not like to deal with chemicals due to residue left on their hands, the smell of the chemicals, etc.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present invention, a device includes a display and a sterilization system. The sterilization system includes an ultra-violet (UV) light source within the device, and the sterilization system is configured to selectively direct UV light from the UV light source into the display.

In some embodiments, the display includes a display surface and an inner surface opposite from the display surface. In certain embodiments, the UV light source is positioned adjacent to the inner surface. In various embodiments, the display includes a side extending between the display surface and the inner surface, and the UV light source is positioned adjacent to the side of the display. In some cases, the UV light source is within the display.

In various embodiments, the UV light source comprises a LV light emitting diode. In some embodiments, the device includes a light emitting diode (LED), and the LED is the UV light source and a display light source. In certain cases, the device includes a first LED and a second LED, the first LED is the UV light source, and the second LED may be a display light source.

In certain embodiments, the device includes a case, and the sterilization system is configured to selectively direct UV light from the UV light source into the case. In various cases, the device is a portable device, and in some embodiments, the device is a mobile phone. IN certain aspects, the UV light source is a first UV light source, and the device comprises a plurality of UV light sources.

According to certain embodiments of the present invention, a device includes an outer surface and a sterilization system within the device relative to the outer surface. The sterilization system includes a UV light source that is configured to selectively direct UV light into at least a portion of the outer surface.

In various embodiments, the device includes a display and a case, the display and the case together define the outer surface of the device, and the sterilization system is configured to selectively direct the UV light into the portion of the outer surface defined by the display. In certain embodiments, the sterilization system is configured to selectively direct the UV light into the portion of the outer surface defined by the case.

In some embodiments, the device is a portable device, and in some cases, the device is a mobile phone. In various aspects, the UV light source is a UV light emitting diode. In some examples, the UV light source is a first UV light source, and the device comprises a plurality of UV light sources.

According to certain embodiments of the present invention, a display for a device includes a glass layer having an inner surface and an outer surface, and a UV light source within the glass layer between the inner surface and the outer surface.

In various embodiments, the UV light source is a first UV light source, and the display comprises a plurality of UV light sources within the glass layer. In some cases, the display also includes a backlight light source. In certain embodiments, the display also includes an organic light emitting diode (OLED) light source.

Various implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 is a perspective view of a device with a sterilization system according to aspects of the disclosure.

FIG. 2 is a sectional schematic of a portion of the device of FIG. 1.

FIG. 3 is a sectional schematic of a portion of another device with a sterilization system according to aspects of the disclosure.

FIG. 4 is a sectional schematic of a portion of another device with a sterilization system according to aspects of the disclosure.

FIG. 5 is a sectional schematic of a portion of another device with a sterilization system according to aspects of the disclosure.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. The shading in the figures is not intended to correspond to any particular material for the components. Rather, the shading has been provided merely to differentiate the different components of the present invention.

Certain embodiments of the present invention provide sterilization systems for surfaces of devices. The sterilization system includes at least one UV light source, such as a UV LED that is integrated or illuminating the device. While reference will be made to UV LED(s), it will be appreciated that various other types of UV light sources may be utilized in addition to or in place of the UV LED(s). The sterilization system is integrated with the device and may be selectively activated such that UV light is emitted to disinfect surfaces of the device. In some examples, the sterilization system is integrated directly into the device such that at least the display (e.g., glass surface) is selectively disinfected upon activation of the UV light. As used herein, an integrated sterilization system refers to one where the UV light source is integrated directly into parts of the device such as the case, backlight, glass cover, etc. An illuminating sterilization system refers to one where the UV light source is mounted as an independent part close to the part of device that is illuminated by the UV light source. The terms “integrated” and “illuminating” are used interchangeably in this description, and reference to only and “integrated” system or an “illuminating” system should not be implied that the description is not applicable to both types of sterilization systems.

The sterilization system may be integrated with the device in a number of ways. As one non-limiting example, the sterilization system may be integrated (or illuminating) the backlight of the display (e.g., for devices or displays that use backlighting like thin-film-transistor (TFT), in-plane switching (IPS), etc.). As another non-limiting example, the sterilization system can be integrated (or illuminating) the display itself. In these examples, the sterilization system may optionally be used for displays or devices that are not using a backlight and/or on displays or devices that are using a backlight but the backlight construction may be UV resistant, may not transfer UV, and/or if some of the layers of the backlight construction absorb too much UV. As another non-limiting example, the sterilization system can be integrated (or illuminating) the cover glass of the display (e.g., Gorilla glass, Cornering glass, Sapphire glass, etc.). In these examples, the sterilization system may optionally be used for devices or displays that are not using a backlight, such as an OLED display, a Q-LED display, an e-paper display, etc. As another non-limiting example, the sterilization system can be integrated (or illuminating) a touch sensor area of the device or display, including but not limited to an additional foil layer and/or thin glass in front of the main display. As another non-limiting example, the sterilization system can be integrated (or illuminating) the shell of the device. In these examples, the shell of the device may be constructed from glass or other suitable transparent materials that do no block UV radiation, and the sterilization system may be used to sterilize the whole device (or sub-portions of the device) such as the display and/or areas without the display such as the backside of the device.

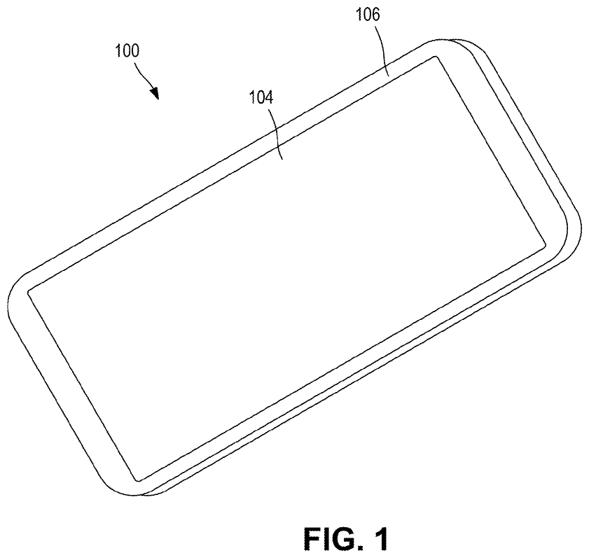

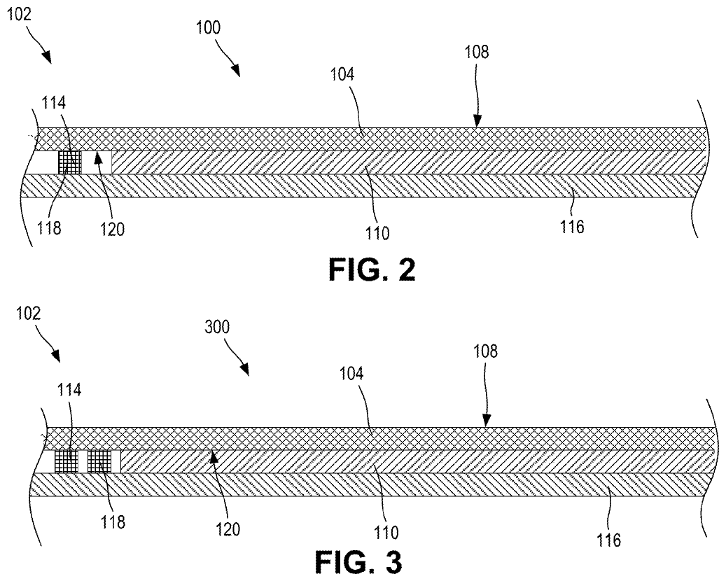

FIGS. 1 and 2 illustrate an example of a device 100 with a sterilization system 102 according to aspects of the current disclosure. The device 100 may be a mobile device (e.g. mobile phone), tablet, personal digital assistants, wearable devices (e.g., smart watches, fitness trackers, etc.), handheld game systems, handheld medical devices with a display, any handheld device with a display, any touchscreen display that may be exposed to different people (e.g., a touchscreen car audio system, an entertainment system, or touchscreen navigation system), or other suitable devices as desired. Accordingly, while the device 100 is illustrated as being portable, in other examples the sterilization system 102 may be provided with a device that is a component of a larger system or structure (such as a navigation system screen) that is not independently portable. Moreover, the particular shape, appearance, and configuration of the device 100 illustrated in FIGS. 1 and 2 should not be considered limiting on the current disclosure.

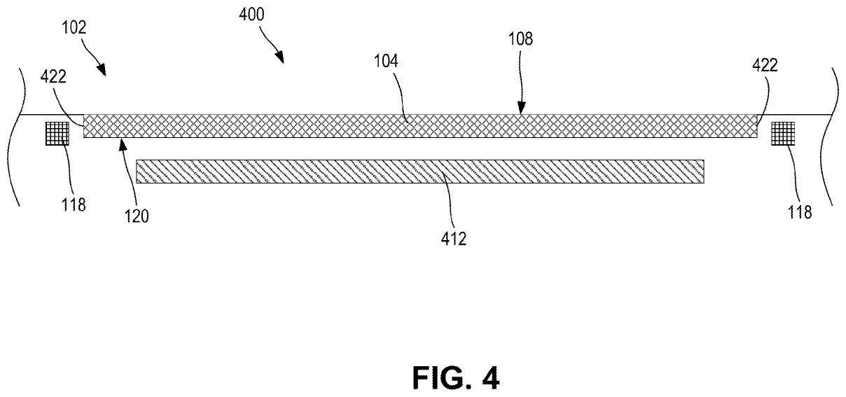

In various examples, the device 100 generally includes a display 104 and a case 106. It will be appreciated that various computing systems and components, communication systems and components, power sources or components, and/or other features of the device 100 may be housed within the case 106. The display 104 generally is a glass layer and has a display (i.e., outer) surface 108. The display 104 may be utilized with other layers depending on the display type (e.g., a backlight display, an OLED display, etc.). The case 106 may be constructed from various suitable materials as desired, including, but not limited to, glass, various metals, plastics, composites, combinations thereof, or other suitable materials. In the example of FIGS. 1 and 2, the display 104 is a component of a backlight display that includes a backlight panel 110. FIG. 4 illustrates an example of a device 400 where the display 104 is a component of an OLED display and includes an OLED layer 412. Various other types of displays may be utilized.

The device 100 may include at least one display light source 114 that selectively illuminates the display 104. The display light source 114 may be supported within the device 100 on a support layer 116 (e.g., a printed circuit board or other suitable layer), although it need not be in other examples. In some examples, the light source is one or more LEDs. The LED(s) may be various types of LEDs including single-die LEDs, multi-die LEDs, direct current (DC) LEDs, alternating current (AC) LEDs, OLEDs, and/or various other suitable LEDs. White, color, or multicolor LEDs, or combinations thereof, may be used. Moreover, the LEDs need not all be the same color and/or type; rather, mixtures of different colors and/or types of LEDs may be used. The LEDs may be provided on one or more printed circuit boards (PCBs). In other embodiments, no PCB is needed; rather the LEDs may be chip-on-board LEDs.

Referring to FIG. 2, the sterilization system 102 includes at least one UV light source 118. In various examples, and as illustrated in FIG. 4 the sterilization system 102 includes a plurality of UV light sources 118. The UV light source 118 may be integrated into the device 100 (e.g., within the device 100 relative to an outer surface of the device 100 defined by the display 104 and/or the case 106). In some cases, the UV light source 118 may be integrated into the display 104, into the case 106, and/or into another component of the device 100. In various cases, the UV light source 118 may be configured to direct light into the component in which it is integrated or into another component. As one non-limiting example, the UV light source 118 may be integrated into the case 106 and may be configured to direct light into the case 106 and/or into the display 104. As another non-limiting example, the UV light source 118 may be integrated into the display 104 and may be configured to direct light into the display 104 and/or into the case 106. In some examples, by integrating the sterilization system 102 into the device (rather than having the UV light source 118 be “outside” the device), the sterilization system does not necessarily need user action to have the process started, the sterilization system does not need another power source, the device with the sterilization system is still compact and is not bulky, and the sterilization system may be fully automated.

In some examples, and as illustrated in FIGS. 1 and 2, the UV light source 118 is a LED. In some examples, and as illustrated in FIG. 2, a single LED may be utilized as both the display light source 114 for the device 100 and the light source 118 for the sterilization system 102. In such examples, the LED may be a dual or multicolor LED, meaning that it can emit UV light and at least one other color light (e.g., white light or other colors as desired). In other examples, the LED provided for the UV light source 118 may be separate from the LED provided for the display light source 114. FIG. 3 illustrates an example of a device 300 that is substantially similar to the device 100 except that the LED provided for the UV light source 118 is separate from the LED provided for the display light source 114.

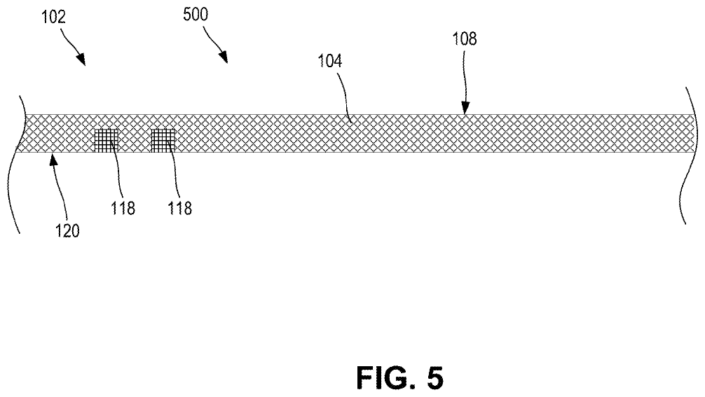

In the examples of FIGS. 1-3, the UV light source 118 is provided adjacent to an inner surface 120 of the display 104 such that the UV light from the UV light source 118 is emitted as a backlight into the display 104. In other examples, the UV light source 118 may be provided at various other locations relative to the display 104 to emit the UV light into the display 104. As one non-limiting example, FIG. 4 illustrates an example of a device 400 where the UV light sources 118 of the sterilization system 102 are arranged such that UV light is emitted into the sides or edges 422 of the display 104 (e.g., into a surface of the edge 422 of the display 104). FIG. 5 illustrates an example of a device 500 where the UV light sources 118 of the sterilization system 102 are integrated directly into the glass of the display 104. In this example, the display 104 with the integrated UV light sources 118 may act like a light pipe. The UV light sources 118 may be integrated into the glass of the display 104 through various suitable techniques as desired. As a further non-limiting example, the UV light source 118 may be spaced apart from the display 104 and may utilize an optical diverter to direct the UV light from the UV light source 118 to the display 104. In other examples, the UV light source(s) 118 may be at other locations relative to the display 104 as desired.

The UV light emitted from the UV light source 118 may disinfect at least the display 104 (e.g., by killing or inactivating microorganisms). In certain examples, and depending on the materials used to form the case 106 (and/or other components of the device), the UV light from the light source 118 may also be used to disinfect other surfaces or components of the device. As one non-limiting example, the case 106 may be constructed from glass, and the UV light from the UV light source 118 may be used to disinfect both the display 104 and the case 106.

In some examples, the UV light source 118 is communicatively coupled to a processor or controller of the device such that the UV light source 118 can be selectively activated or deactivated as desired. In other examples, the sterilization system 102 may have a separate controller or processor is communicatively coupled with the UV light source 118 and that selectively controls the activation and deactivation of the UV light source 118 as desired. When the sterilization system 102 includes its own controller or processor, the controller or processor may be provided at the same location as the UV light source 118 (e.g., at a same general location on the device 100), although in other embodiments, the controller of the sterilization system 102 may be provided at a location that is different from the location of the UV light source 118.

In certain aspects, the sterilization system may optionally be controlled (e.g., by a controller of the device 100, a controller of the sterilization system 102, and/or by a user) such that the UV radiation is provided only under certain conditions. As one non-limiting example, the sterilization system may be controlled such that there is no or minimal UV radiation when the device is exposed to the user's eyes. In such examples, the sterilization system optionally includes a sensor that may detect one or more operating condition of the device (e.g., detects whether the display is covered due to a phone cover or in a protection pocket or otherwise covered). The sensor may be communicatively coupled to one or more components of the sterilization system such that the UV process starts or is able to start based on a suitable operating condition. The sensor may also be communicatively coupled to one or more components of the sterilization system to interrupt or otherwise control the UV process based on a change in operating condition(s).

A collection of exemplary embodiments, including at least some explicitly enumerated as “Illustrations,” providing additional description of a variety of embodiment types in accordance with the concepts described herein are provided below. These examples are not meant to be mutually exclusive, exhaustive, or restrictive; and the invention is not limited to these example embodiments but rather encompasses all possible modifications and variations within the scope of the issued claims and their equivalents.

Illustration 1. A device comprising: a display; and a sterilization system comprising a UV light source, wherein the sterilization system is configured to selectively direct UV light from the UV light source into the display.

Illustration 2. The device of any preceding or subsequent examples or combination of illustrations, wherein the display comprises a display surface and an inner surface opposite from the display surface, and wherein the UV light source is positioned adjacent to the inner surface.

Illustration 3. The device of any preceding or subsequent examples or combination of illustrations, wherein the display comprises a display surface, an inner surface opposite from the display surface, and a side extending between the display surface and the inner surface, and wherein the UV light source is positioned adjacent to the side of the display.

Illustration 4. The device of any preceding or subsequent examples or combination of illustrations, wherein the UV light source is within the display.

Illustration 5. The device of any preceding or subsequent examples or combination of illustrations, wherein the UV light source comprises a UV light emitting diode.

Illustration 6. The device of any preceding or subsequent examples or combination of illustrations, further comprising a LED, and wherein the LED is the UV light source and a display light source.

Illustration 7. The device of any preceding or subsequent examples or combination of illustrations, further comprising a first LED and a second LED, wherein the first LED is the UV light source and wherein the second LED is a display light source.

Illustration 8. The device of any preceding or subsequent examples or combination of illustrations, further comprising a case, and wherein the sterilization system is configured to selectively direct UV light from the UV light source into the case.

Illustration 9. The device of any preceding or subsequent examples or combination of illustrations, wherein the device is a mobile phone.

Illustration 10. The device of any preceding or subsequent examples or combination of illustrations, wherein the UV light source is a first UV light source, and wherein the device comprises a plurality of UV light sources.

Illustration 10a. The device of any preceding or subsequent examples or combination of illustrations, wherein the device is a portable device.

Illustration 10b. The device of any preceding or subsequent examples or combination of illustrations, wherein the portable device is a mobile phone.

Illustration 11. A device comprising: an outer surface; and a sterilization system within the device relative to the outer surface, the sterilization system comprising a UV light source that is configured to selectively direct UV light into at least a portion of the outer surface.

Illustration 12. The device of any preceding or subsequent examples or combination of illustrations, wherein the device comprises a display and a case, wherein the display and the case together define the outer surface of the device, and wherein the sterilization system is configured to selectively direct the UV light into the portion of the outer surface defined by the display.

Illustration 13. The device of any preceding or subsequent examples or combination of illustrations, wherein the sterilization system is configured to selectively direct the UV light into the portion of the outer surface defined by the case.

Illustration 14. The device of any preceding or subsequent examples or combination of illustrations, wherein the device is a portable device.

Illustration 14a. The device of any preceding or subsequent examples or combination of illustrations, wherein the portable device is a mobile phone.

Illustration 15. The device of any preceding or subsequent examples or combination of illustrations, wherein the UV light source is a UV light emitting diode.

Illustration 16. The device of any preceding or subsequent examples or combination of illustrations, wherein the UV light source is a first UV light source, and wherein the device comprises a plurality of UV light sources.

Illustration 17. A display for a device comprising: a glass layer comprising an inner surface and an outer surface; and a UV light source within the glass layer between the inner surface and the outer surface.

Illustration 18. The display of any preceding or subsequent examples or combination of illustrations, wherein the UV light source is a first UV light source, and wherein the display comprises a plurality of UV light sources within the glass layer.

Illustration 19. The display of any preceding or subsequent examples or combination of illustrations, further comprising a backlight light source.

Illustration 20. The display of any preceding or subsequent examples or combination of illustrations, further comprising an OLED light source.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

1. A device comprising:
a display; and
a sterilization system comprising an ultra-violet (UV) light source within the device, wherein the sterilization system is configured to selectively direct UV light from the UV light source into the display.

2. The device of claim 1, wherein the display comprises a display surface and an inner surface opposite from the display surface, and wherein the UV light source is positioned adjacent to the inner surface.

3. The device of claim 1, wherein the display comprises a display surface, an inner surface opposite from the display surface, and a side extending between the display surface and the inner surface, and wherein the UV light source is positioned adjacent to the side of the display.

4. The device of claim 1, wherein the UV light source is within the display.

5. The device of claim 1, wherein the UV light source comprises a UV light emitting diode.

6. The device of claim 1, further comprising a light emitting diode (LED), and wherein the LED is the UV light source and a display light source.

7. The device of claim 1, further comprising a first LED and a second LED, wherein the first LED is the UV light source and wherein the second LED is a display light source.

8. The device of claim 1, further comprising a case, and wherein the sterilization system is configured to selectively direct UV light from the UV light source into the case.

9. The device of claim 1, wherein the device is a mobile phone.

10. The device of claim 1, wherein the UV light source is a first UV light source, and wherein the device comprises a plurality of UV light sources.

11. A device comprising
an outer surface; and
a sterilization system within the device relative to the outer surface, the sterilization system comprising an ultra-violet (UV) light source that is configured to selectively direct UV light into at least a portion of the outer surface.

12. The device of claim 11, wherein the device comprises a display and a case, wherein the display and the case together define the outer surface of the device, and wherein the sterilization system is configured to selectively direct the UV light into the portion of the outer surface defined by the display.

13. The device of claim 12, wherein the sterilization system is configured to selectively direct the UV light into the portion of the outer surface defined by the case.

14. The device of claim 11, wherein the device is a mobile phone.

15. The device of claim 11, wherein the UV light source is a UV light emitting diode.

16. The device of claim 11, wherein the UV light source is a first UV light source, and wherein the device comprises a plurality of UV light sources.

17. A display for a device comprising
a glass layer comprising an inner surface and an outer surface; and
an ultra-violet (UV) light source within the glass layer between the inner surface and the outer surface.

18. The display of claim 17, wherein the UV light source is a first UV light source, and wherein the display comprises a plurality of UV light sources within the glass layer.

19. The display of claim 17, further comprising a backlight light source.

20. The display of claim 17, further comprising an organic light emitting diode light source.
